(19) United States
(12) Patent Application Publication
(10) Pub. No.: US 2018/0338866 A1
(43) Pub. Date: Nov. 29, 2018

(54) Electroactive dressings

(71) Applicant: Mohammad Ali Kharazmi, Corona Del Mar, CA (US)

(72) Inventor: Mohammad Ali Kharazmi, Corona Del Mar, CA (US)

(21) Appl. No.: 15/927,988

(22) Filed: Mar. 21, 2018

Related U.S. Application Data
(60) Provisional application No. 62/474,575, filed on Mar. 21, 2017.
(60) Provisional application No. 62/510,755, filed on May 25, 2017.

(51) Int. Cl. A61F 13/02; A61L 24/00; A61F 13/84; H01B 1/12

(52) U.S. Cl. CPC A61F 13/02; A61L 24/0031; A61K 31/352; H01B 1/124; A61F 13/84

(57) ABSTRACT

A dressing for treating a wound includes a flexible membrane having an upper surface and a wound-facing surface, a flexible battery, an electrode pattern to deliver current to the wound, and a current control electrically to limit the amount of current delivered to the electrode pattern. In embodiments, the current control includes active circuits, series resistors, and parallel resistors. A method of treating a wound includes applying the described dressing and adjusting the current. Embodiments include applying a conductive gel containing a stem cell culture medium or cannabidiol.

### RELATED PATENT APPLICATION

This application claims the benefit of U.S. provisional patent application No. 62/474,575, filed on Mar. 21, 2017. This application claims the benefit of U.S. provisional patent application No. 62/510,755, filed on May 25, 2017. The entire content of the provisional patent applications are incorporated herein in their entireties for all purposes.

### FIELD OF THE INVENTION

This invention is in the field of dermal care and treatment. In particular, it concerns electroactive dressings and bandages that improve the condition of wounds, scars, or burns.

### BACKGROUND OF THE INVENTION

Bandages for wounds or burns are commonly composed of sterile absorbent dressings that are fastened in place by separate fasteners such as tape, adhesives, compressive textiles, or ties. Some bandages may be pretreated with antimicrobials to retard wound infection. For example, wound dressings impregnated with certain healing promoting or microbiocidal materials, such as nanosilver, cause wounds to heal more quickly. Other bandages may be untreated but applied with or over topically applied aids such as antimicrobials, clotting factors, and desiccants.

Numerous publications report that healing of skin wounds is stimulated by electrical current. See, for example, O M Alvarez in J Investigative Dermatology 81, 1983; pp. 144-148. T A Banks, et al. reported in Integr. Biol., 2015; 7, pp. 693-712 that human bone marrow-derived mesenchymal stem cells migrate in response to applied electric fields. The authors wrote regarding the significant regenerative potential in the observed improved healing in vivo post applied electric fields and that the intrinsic piezoelectric nature of collagenous-rich tissues, such as bone and cartilage, can result in the production of small, endogenous electric fields during applied mechanical stresses. H H Park et al. reported in Appl. Phys. Lett. 105, 2014; 24, p 4109 that induced electric fields could control directional migration of rat mesenchymal stem cells. The authors observed mesenchymal stem cell migration during wound closure in presence of an indirect electric field. B Vanhaesebroeck in Nature Chemical Biology 2, 2006; pp. 453-455 reported that manipulation of electric fields affect wound healing in vivo and identified the phosphoinositide 3-kinase signaling pathway as a key component of cell migration in response to electric cues.

Electroactive wound dressings produce local electric fields by providing electrical half cells in proximity to healing skin. Wound exudate or exogenously administered fluid close the half cells into a full electrical cell, or battery of electrical cells that generate low-level currents between electrodes on the dressing and extending into proximal healing tissue. Commercially available dressings sold by Vomaris Wound Care, Inc. of Tempe Ariz. under the registered trademark Procellera® are said to provide effective antimicrobial protection to the wound site, inhibiting the growth of harmful microorganisms that may cause infection. Without infection, wounds are said to heal faster. The dressings feature a staggered matrix pattern of silver or silver chloride and zinc electrodes dots applied to the dressing surface.

The current delivered by such dressings depends on the potential of the half cells included in the dressing, on the geometry of the dressing and its contact with the wound, on the characteristics of the fluid in contact with the dressing, and on the structure of the wounded tissue.

Other wound dressings rely on external sources of current. For example, U.S. Pat. No. 6,907,294 to Andino et al. is said to disclose an electrode system that generates a current flow that envelops and permeates an entire wound site. The electrode system includes two electrodes shaped as an annulus surrounding a central spot and causing current to flow through the wound. A power supply, which may be local to or remote from the electrode system, applies a voltage potential across the electrodes. Alternatively, U.S. Pat. No. 6,907,294 to Andino et al. also describes an embodiment where the two electrodes are unspecified oppositely-charged polymers that are said to cause a current to flow between the electrodes without an external power supply or leads.

While externally driven electroactive dressings provide the ability to preset a desired amount of current (or voltage) to be applied to a wound, this requires connection to external appliances that interfere with or hinder patient mobility. In some circumstances the weight of attached leads or devices may cause a dressing to peel from a treatment site, exposing a wound or causing a less than desired degree of treatment. There is thus a need to provide an improved electroactive dressing where the applied current is controllable without the weight and inconvenience of attached wires or external devices. Further, a battery that is rigid and heavy may cause discomfort to the treated individual and may also cause a dressing to peel or detach from a dressing site when the skin is flexed. There is thus a need to provide an improved electroactive dressing with a flexible power source that does not cause discomfort or detachment.

Setting the desired current may be complex or may require tools not normally available when dressings are applied or changed. There is thus a need to provide an improved electroactive dressing where the current applied to a wound is controllable at the time of application without specialized tools.

The desired current applied to a wound may vary over the time course of treatment. Replacement of a dressing in intimate contact with healing skin may disrupt the treated surface. There is thus also a need to provide a dressing where the amount of current may be changed during the course of treatment without recourse to specialized tools.

### SUMMARY

In embodiments, the invention includes a dressing for treating a wound, where the dressing includes a flexible membrane, a battery, an electrode pattern, and a current control. The flexible membrane has an upper surface and a wound-facing surface opposing the upper surface. The battery may be affixed to the upper surface, and the electrode pattern may be disposed on the wound-facing surface. The electrode pattern delivers current to the wound. The current control is electrically connected to the battery and to the electrode pattern and is configured to limit the amount of current delivered to the electrode pattern.

In embodiments, the current control is configured to select the amount of current delivered to the electrode pattern. The current control may include one or more of an active circuit, a series resistance control, or a parallel resistance control. The current control may include a plurality of resistors arranged in a parallel circuit between the battery and the electrode pattern, where the dressing has a trim edge and the plurality of resistors are disposed in a spaced-apart relationship and parallel to the trim edge.

In other embodiments, the current control includes a plurality of resistors arranged in a series circuit between the battery and the electrode pattern. The upper surface may include a first contact electrically connected to the electrode pattern and a plurality of resistor contacts, with each resistor contact electrically connected to a respective one of the plurality of resistors. A bridging contact may be configured to electrically connect the first contact to a selected one of the plurality of resistor contacts.

The electrode pattern may include interdigitated electrodes, such as a sawtooth interdigitated electrode. The battery may be one of a printed battery and a laminated battery.

The invention also includes a method of treating a wound including steps of adjusting the current delivered by the dressing as described and applying the dressing to the wound to be treated. When the current control includes a plurality of resistors disposed in a spaced-apart relationship parallel to a trim edge and arranged in a parallel circuit between the battery and the electrode pattern, the step of adjusting the current may include cutting the dressing parallel to the trim edge such that one or more of the plurality of resistors are removed from the parallel circuit.

In other embodiments, the current control may include a plurality of resistors arranged in a series circuit between the battery and the electrode pattern. The upper surface may include a first contact electrically connected to the electrode pattern, a plurality of resistor contacts, and a bridging contact. Each resistor contact may be electrically connected to a respective one of the plurality of resistors. The bridging contact may be configured to electrically connect the first contact to a selected one of the plurality of resistor contacts. The step of adjusting the current in such embodiments includes folding the dressing to electrically connect the bridging contact between the first contact and a selected one of the plurality of resistor contacts so that one or more of the resistors may be shunted out of the series circuit.

In embodiments, the method includes readjusting the current delivered by the dressing after applying the dressing and applying a conductive treatment gel including an HADSCC media and a gelling agent between the dressing and the wound.

The invention also includes a kit including the dressing as described and a conductive gel including an HADSCC media and a gelling agent, where the gel has a viscosity of at least 3000 cP.

### BRIEF DESCRIPTION OF THE DRAWINGS

FIG. 1a illustrates a perspective view of an embodiment of the dressing of the invention.

FIG. 1b illustrates a side view of the embodiment of FIG. 1a.

FIG. 2a illustrates a first embodiment of an electrode pattern of the dressing of the invention.

FIG. 2b illustrates a second embodiment of an electrode pattern of the dressing of the invention.

FIG. 3 illustrates a schematic of an active circuit current control of an embodiment of the dressing of the invention.

FIG. 4 illustrates an embodiment of the dressing of the invention with a parallel resistance current control.

FIG. 5 illustrates an embodiment of the dressing of the invention with a series resistance current control.

### DETAILED DESCRIPTION

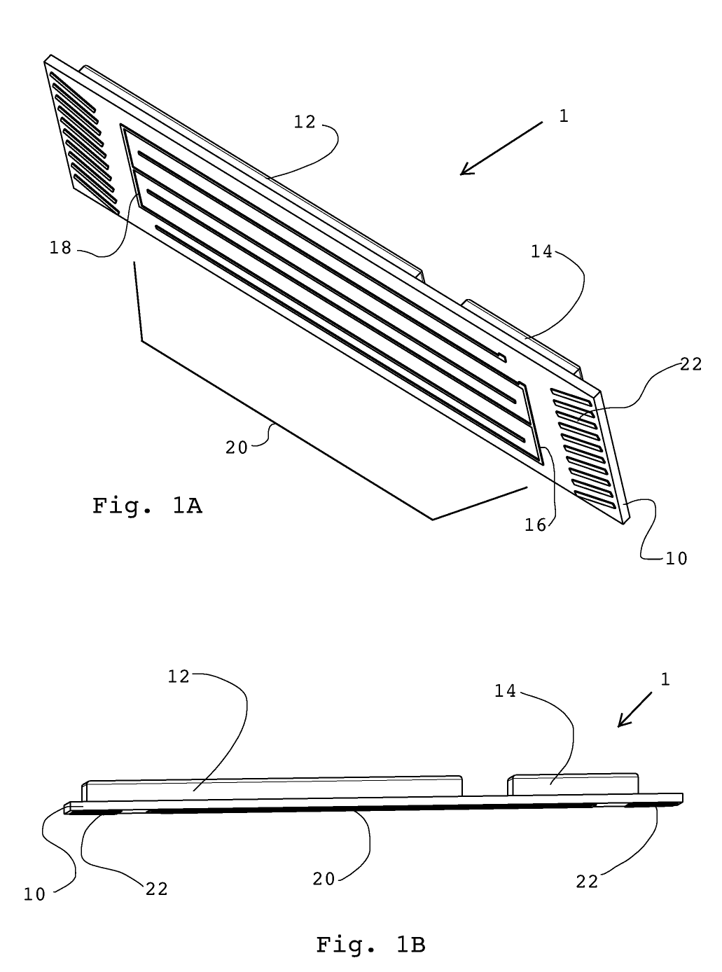

Referring to FIGS. 1a and 1b, an embodiment of the dressing 1 of the invention is substantially planar and has an upper surface and a lower, wound-facing surface. Dressing 1 may be sterilized prior to use and may be packaged to maintain sterility. Dressing 1 includes a flexible membrane 10 supporting an electrode pattern 20, a battery 12, and a current controller 14.

The flexible membrane 10 may be any substantially planar flexible material that is not water soluble. The membrane may be either permeable or impermeable depending on the particular application. Permeable materials allow a dressed wound to exchange gasses and humidity with the surrounding space. Impermeable materials isolate the wound. Absorbent materials may advantageously absorb wound fluids or provide treatment fluids. Suitable materials include paper or other cellulosic materials, polymers such as polyethylene, silicone, or acrylic, and other materials. The material may be relatively thin with respect to its extent to flex easily with movement. Thicknesses of about 0.001 to about 0.050 inch may be suitable. Flexible membrane 10 may be laminated of more than one layer of material to achieve a desired mix of properties, such as an absorbent cellulosic layer laminated to one or more polymer layers to provide strength and conduction paths. Flexible membrane 10 provides benefits of supporting the other components of the dressing in proximity to the treated surface, even if the treated surface bends and flexes during use.

In extent, the flexible membrane defines the size of the dressing. Suitable sizes may be formed to fit a variety of wound areas and shapes, much as in conventional dressings.

Flexible membrane 10 may include features that secure dressing 1 to the wounded surface. These features may include adhesive 22 applied to all or part of the wound-facing surface. Adhesives may be conventional dressing adhesives such as acrylics or may be hydrogels that optionally cover the entire wound-facing surface. In other embodiments, the flexible membrane may be attached to the wound by external compressive strips or tape. A benefit of adhesive 22 is that it holds the dressing in place without additional materials or parts. However, the geometry of some wounds may be more compatible with external ties such as compressive strips or tape.

Flexible membrane 10 may include conductive traces on one or both planar surfaces. Conductive traces may be formed as deposited inks in an additive process or may be etched through a plated conductor foil similar to construction of a printed circuit board. Traces may be fully conductive or may be resistive, using any of a variety of resistive inks comprising carbon particles in a binder. Resistive traces may form part or all of current controller 14. Conductive traces applied to the wound-facing surface of flexible membrane 10 may form electrode pattern 20. Traces may communicate between upper surface and wound-facing surface through vias, by extending around edges of flexible membrane 10, or by forming conductive traces on a single surface and folding flexible membrane 10 with the conductive traces along a midline to reflect a portion of the surface with traces onto the wound-facing surface. In such embodiments, the adjacent surfaces of flexible membrane 10 may be held with an adhesive.

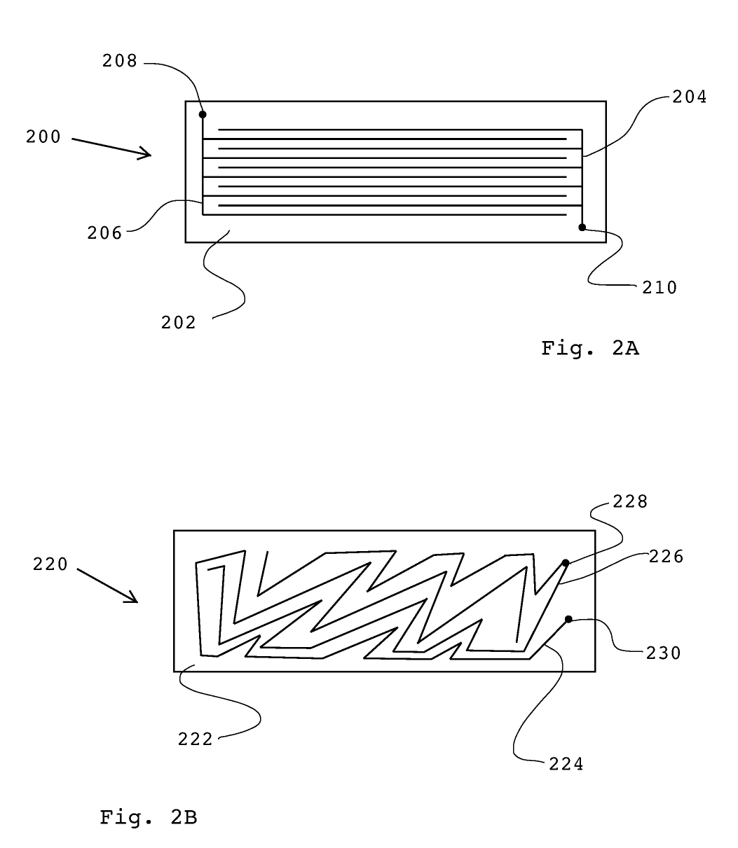

The electrode pattern 20 is a collection of conductors 16 and 18 disposed on the wound-facing surface. Each of conductors 16 and 18 communicates (directly or indirectly) with one terminal of battery 12. In a suitable environment, such as when dressing 1 is in contact with a wound in the presence of a conducting liquid (such as wound exudate or an applied conductive gel), current flows between conductors 16 and 18. Conductors 16 and 18 are arranged in pattern 20 to distribute current at least partially into the wound. Suitable patterns 20 include a variety of spaced-apart conductors, such as linear interdigitating electrodes 204 and 206 of dressing 200 of FIG. 2a. Conductor 204 feeds through membrane 202 to current control (not visible) at via 210. Similarly, conductor 206 feeds through membrane 202 to current control (not visible) at via 208.

FIG. 2b illustrates an alternative embodiment showing a sawtooth pattern of interdigitating electrodes 224 and 226 of dressing 220. Conductor 224 feeds through membrane 222 to current control (not visible) at via 220, and conductor 226 feeds through at via 228. Sawtooth pattern includes relatively sharp corners which may “sculpt” the electric fields between the corners and the counterelectrode, thereby allowing a degree of regulation of the distribution of current through the tissue. In some embodiments (not illustrated) the conductors may be arranged to deliberately deliver more current to some regions of a wound than to other regions. This may be appropriate when the severity of the wound varies over the treated surface.

A large variety of patterns may be suitable for electrode pattern 20. This includes interdigitating electrodes as described above, other interdigitating patterns, discrete electrodes such as circular electrodes with concentric annular counter-electrodes, or patterns of discrete electrodes connected by conductive traces on a buried face of flexible membrane 10, among others.

Battery 12 may be any of a variety of thin flexible batteries such as printed batteries or laminated batteries. Battery 12 may be flexible to move with flexible membrane 10 of dressing 1, advantageously keeping dressing 1 in contact with a wound during movement. Battery 12 may be formed directly upon flexible membrane 10 or may be separately formed and adhered by adhesive, tapes, slits, folds, or clips.

Thin flexible electrical batteries are known in the art. For example, U.S. Pat. No. 7,320,845 to Zucker describes a printed battery having a flexible backing sheet, a first conductive layer printed on the sheet; a first electrode printed on the first conductive layer; a second electrode layer printed on the first electrode layer; and a second conductive layer printed on the second electrode layer. A wide variety of cell chemistries are disclosed, including Leclanché (zinc-anode, manganese dioxide-cathode), Magnesium (Mg-anode, MnO₂-cathode), Alkaline MnO₂ (Zn-anode, MnO₂-cathode), Mercury (Zn-anode, HgO-cathode), Mercad (Cd-anode, Ag₂O-cathode), and Li/MnO₂(Li-anode, MnO₂-cathode). Particles of the electrode materials material are mixed into an ink base and applied to paper or to a sheet of polyester film.

Other flexible batteries may be made of laminated foils and membranes, by vacuum deposition, sputtering, ion-plating, or non-electrolytic plating, or by combinations of the above methods and materials, including that described in U.S. Pat. No. 8,268,475 to Tucholski. Such batteries include Zinc-Manganese Dioxide primary cells as thin as 0.025 inches. Any of the above described batteries, and others that have similar properties and dimensions, may serve as battery 12 of dressing 1. In one embodiment, a commercial battery marketed by Blue Spark Technologies of Westlake, Ohio may be glued to flexible membrane 10 using an acrylic adhesive or double-sided tape. Leads from battery 12 may couple to conductive traces on the surface of flexible membrane 10. In some embodiments, battery 12 and flexible membrane 10 may receive a protective overcoating or lamination to hold the assembly together and to protect conductive traces

The current controller controls the current delivered to the treated area. This advantageously allows modification of treatment as appropriate for the individual injury as well as providing a more consistent treatment regimen.

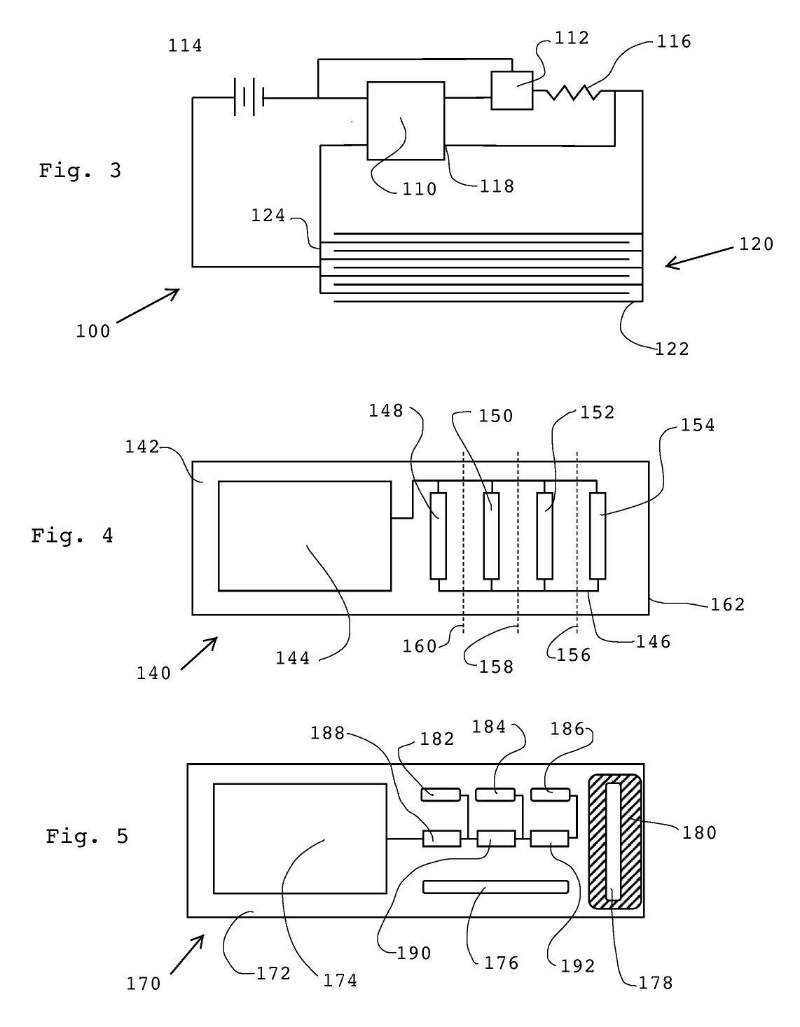

In some embodiments, such as that schematically illustrated in FIG. 3, the current controller 100 may include an active device, such as a processor or microcontroller 110 driving a pulse width modulator 112 delivering current to the electrode pattern 120 (including interdigitated electrodes 122 and 124). Microcontroller 110 may be any of a variety of low cost single-chip microcontrollers, such as a PIC10F204 produced by Microchip Technology of Chandler, Ariz. Microcontroller 110 may include feedback elements such as a series resistor 116 with voltage sampled by the microcontroller at 118. Pulse width modulator 112 may include a driver such as a DRV8837 H-Bridge driver produced by Texas Instruments of Dallas, Tex. Microcontroller 110 may adjust the duty cycle of pulse width modulator 112 to produce the desired level of current as measured by the drop across resistor 116 in series with the electrode pattern. The electronic components may be encapsulated in a polymer or packaged in a fluid tight compartment to prevent degradation from the wound or the ambient environment. Microcontroller 110 may be activated and may receive current set commands through any of a number of methods known in the art such as Bluetooth or other radio communication, optical communication, or one or more switches such as membrane switches packaged with the microcontroller and accessed through pressure contact with the compartment.

In some embodiments, microcontroller 110 may receive current set commands through its input ports. Conductors (not illustrated) may electrically connect to ports of microcontroller 110. The other end of these conductors may be connected to a programming source, which may be, for example, either terminal of the battery or an output port of microcontroller 110. The current set command may be determined by which input ports are connected to the programming source via the conductors. The connections may be chosen by the user by selectively cutting one or more of the conductors. In one embodiment, the conductors may be spaced apart from one another and arrayed parallel to a trim edge of the dressing (as described below with respect to the resistors of embodiment of FIG. 4). A user selects the desired current program by trimming the dressing between conductors parallel to the trim edge. This severs the conductors that are closer to the trim edge than the cut, removing the respective connection of programming source to input port. The input ports may include pull up or pull down resistors to keep the inputs in a stable configuration if corresponding conductors are severed.

In other embodiments, conductors may be selectively connected to input ports by folding the dressing so that one or more conductors attach it to a contact pad electrically coupled to a programming source. This is similar to the process described below for the series resistor embodiment of FIG. 5.

The above disclosed active component control dressings advantageously allow selection of the desired current without special tools or devices other than scissors or similar cutting tools commonly available. A single dressing of this type may have its current changed as the wound improves by additional trimming. A method of using the dressing includes steps of applying the dressing to a wound for a first amount of time and trimming the dressing to change programmed current while the dressing remains in position. This adjustment advantageously permits “tuning” of the applied current as appropriate to different stages of healing.

Because microcontroller 110 accepts complex programming, the changes in programmed current with successive trimming may be arbitrary—there need be no fixed relationship between current programming from successive trims. The change in current may be nonlinear and need not be monotonic.

In other embodiments, the current may rely on passive component control, such as introducing a selectable resistance between the battery and the electrode pattern. In some embodiments, each dressing may include a single set value of resistance. One or more resistors may be printed or discretely applied. A user may select among several different dressings each with a different set value of resistance to select the desired current.

In other embodiments, a single dressing may have an adjustable resistance. In a parallel resistor embodiment of FIG. 4, a dressing 140 includes several resistors 148, 150, 152, and 154 disposed in parallel (which may be printed or discretely applied). The parallel resistors may be arrayed parallel to one edge 162 of the dressing. A user selects the desired current by trimming the dressing between resistors parallel to the edge as marked at 162 at 156, 158, or 160. The intact dressing has the lowest resistance and hence delivers the highest current. Removal of one or more resistors by trimming the dressing 140 increases the remaining resistance. In such embodiments, the dressing may include printed lines 162 at 156, 158, and 160 indicating where the dressing should be trimmed to achieve the desired current. The value of the resistors may be equal or may be chosen such that each trim produces a desired decrease in current. A single dressing of this type may have its current reduced as the wound improves by additional trimming. A method of using the dressing 140 includes steps of applying the dressing to a wound for a first amount of time and trimming the dressing to increase resistance while the dressing remains in position. This adjustment advantageously permits “tuning” of the applied current as appropriate to different stages of healing.

In a series resistor embodiment of FIG. 5, a dressing 170 includes several resistors 188, 190, and 192 disposed in series with electrical contact pads 182, 184, and 186 between the resistors. An elongated contact pad 176 may connect to one side of the electrode pattern (not shown). A conductive shorting strip 178 near one end of a surface (such as the upper surface) of the dressing may be reflected back to selectively short one of resistor contact pads 182, 184, and 186 to electrode contact pad 176, selecting the desired resistance. Adhesive 180 surrounding the shorting strip may hold the reflected end in place.

The above disclosed passive component control dressings advantageously allow selection of the desired current without special tools or devices while maintaining a low cost of the dressings.

In other embodiments the invention includes a dressing as described above and including a conductive gel composition including a conditioned cell culture medium. Many tissues, including living layers of the skin, respond to appropriate mixtures of growth factors to encourage regeneration. PCT US2014/034738 describes dermal treatment compositions including a medium recovered from human adipose-derived stem cell culture (HADSCC). HADSCC produce a variety of growth-promoting and healing materials such as growth factors, cytokines, stress proteins, and nutrients including TGF-B, PDGF, and GM-CSG, interleukins, and matrix proteins (collectively, stem cell products). While many of these have been identified, the cells likely also secrete other substances due to their pluri-potency either not yet known or with beneficial functions yet to be precisely identified. Some of these materials may be effective at low concentration.

The conductive gel composition contains HADSCC media at about 50% by weight of the composition. The gel also may include a gelling agent and a viscosity of at least 3000 cP. In embodiments, the gelling agent may be at least 1% by weight of the gel. The gelling agent may be a hydroxymethyl cellulose or a carboxymethyl cellulose. The conductive gel may include ionic salts from about 50 mEq/L to about 200 mEq/L and in some embodiments about 140 mEq/L and may optionally include a nanosilver particulate.

The conductive gel composition may be applied to the wound-facing surface of dressing 1 prior to application of the dressing to the wound. The gel serves as a conductive medium that electrically couples the electrode pattern to the tissue and delivers the current produced by dressing 1. In other embodiments, other conductive gels or liquids may be employed, or wound exudate may serve to couple the current.

The above described gel may advantageously deliver healing-promoting growth factors and cell products to a wound.

In other embodiments, the invention includes an electroactive dressing as described above in conjunction with a conductive gel composition including cannabidiol.

Cannabidiol is the major nonpsychoactive ingredient in cannabis. It is reported to possess neuroprotective and anti-inflammatory effects. The major psychotropic component of cannabis, Δ⁹-THC, activates the endocannabinoid system, which consists of receptors, synthetic and degradative enzymes, and transporters. Δ⁹-THC binds to two G-protein-coupled cell membrane receptors, consequently named the G-protein-coupled cannabinoid (CB) cannabinoid type 1 (CB₁) and type 2 (CB₂) receptors, to exert its effects. Endogenous lipophilic ligands (endocannabinoids) including anandamide and 2-arachidonoylglycerol also bind CB₁ and CB₂. CB₁ receptors are found primarily in the brain but also in several peripheral tissues. CB₂ receptors are mainly found in immune and hematopoietic cells, but can become upregulated in other tissues.

Cannabidiol has low affinity for CB₁ and CB₂ receptors, but appears to act as both an agonist and antagonist of CB₂ receptors depending on concentration. This paradoxical action is likely due to indirect action on other receptors or enzymes that are functionally linked to the CB₂ receptor. Cannabidiol interacts with many other, non-endocannabinoid signaling systems. At low micromolar to sub-micromolar concentrations, cannabidiol blocks equilibrative nucleoside transporter (ENT), the orphan G-protein-coupled receptor GPR55, and the transient receptor potential of melastatin type 8 (TRPM8) channel. Conversely, cannabidiol enhances the activity of the 5-HT₁ₐ receptor, the α3 and α1 glycine receptors, the transient receptor potential of ankyrin type 1 (TRPA1) channel, and has a bidirectional effect on intracellular calcium. At higher micromolar concentrations, cannabidiol activates the nuclear peroxisome proliferator-activated receptor-γ and the transient receptor potential of vanilloid type 1 (TRPV1) and 2 (TRPV2) channels while also inhibiting cellular uptake and fatty acid amide hydrolase-catalyzed degradation of anandamide. Cannabidiol is a potent antioxidant because of its multiple phenolic structures.

Cannabidiol has high lipophilicity (K_{octanol-water}^{˜}6-7) and consequently very low water solubility. This limits its availability in many formulations. In embodiments, the invention increases the effective solubility of cannabidiol by associating the cannabidiol with other agents. While these other agents may be simple hydrophobic solvents such as octanol, such solvents are also sparingly soluble in water unless emulsified. In embodiments, cannabidiol is co-solubilized by mixing into liposomes containing one or more of lipophilic surfactants such as dipalmitoylphosphatidylcholine or phosphatidylinositol. Cholesterol may be added as a further liposome component to improve stability. Dipalmitoylphosphatidylcholine is a phospholipid consisting of two palmitic acids attached of a phosphatidylcholine head-group. Phosphatidylinositol is a phosphatidylglyceride including an inositol group. These materials are merely illustrative of a class of lipophilic surfactants such as occur in cell membranes. These materials have been reported to be useful to prepare liposomes containing cannabidiol (see, for example, Hung, et al. PCT publication WO 01/03668, incorporated by reference for its teaching of liposome encapsulation of cannabinoids). One or more of these lipophilic surfactant material (or a mixture of the materials with cholesterol) may be mixed with cannabidiol in organic solvent with cannabidiol forming between 0.5 and 10% of the weight of the mixture. After drying the solvent the residue may be mixed with phosphate buffered saline (120 mM, pH 7) and extruded through 400 nm pore sized polycarbonate filters to form liposomes.

In some embodiments, a lower concentration of dipalmitoylphosphatidylcholine or phosphatidylinositol may be used so that each cannabidiol molecule is paired with one to five lipophilic surfactant molecules below the critical micellar concentration and without extrusion of liposomes. In still other embodiments, cannabidiol may be combined with a protein such as human serum albumin to act as a carrier molecule. Each of these materials added together with cannabidiol to aqueous mixtures or suspension will be referred to as co-solubilizers.

The benefit of the co-solubilization of cannabidiol is that more cannabidiol may be delivered in a substantially aqueous mixture. A further benefit is that the co-solubilized cannabidiol may gradually extract from its co-solubilizer, making the cannabidiol available in solution over an extended time. A still further benefit that the co-solubilized material acts as a reservoir to “buffer” the cannabidiol concentration in the aqueous phase to a relatively constant sustained value.

Cannabidiol suppresses interleukin (IL) 8 and 10 production and induces lymphocyte apoptosis in vitro. It is a strong inhibition of neutrophil chemotaxis and modulates tumor necrosis factor (TNF)-α, IL-1, and interferon (IFN)-γ by mononuclear cells and the suppression of chemokine production by human B cells. Cannabidiol's overall effect is generally considered anti-inflammatory, though its suppression of the anti-inflammatory IL-10 suggests more complex effects. Schmuhl et al. (in Biochemical Pharmacology, 87: 3 pp 489-501 (2014)) reported an increase of mesenchymal stem cell migration by cannabidiol via activation of p42/44 MAPK. Migration and differentiation of mesenchymal stem cells (MSCs) are known to be involved in various regenerative processes such as bone healing. Cannabidiol was reported to increase the migration of adipose-derived MSCs in a time-and concentration-dependent manner. Endocannabinoid (eCB) signaling has also been shown to regulate proliferation and differentiation of mesoderm-derived hematopoietic and mesenchymal stem cells, with a key role in determining the formation of several cell types in peripheral tissues, including blood cells, adipocytes, osteoblasts/osteoclasts and epithelial cells. Long-term stimulation with cannabidiol induced differentiation of MSCs into the osteoblastic lineage as evidenced by increased mineralization. Cannabidiol may therefore recruit MSCs to sites of calcifying tissue regeneration and subsequently support bone regeneration.

Applicant has found that cannabidiol may have beneficial effects when applied in a conductive gel with electroactive dressings. The gel also may include a gelling agent having a viscosity of at least 3000 cP. In embodiments, the gelling agent may be at least 1% by weight of the gel. The gelling agent may be a hydroxymethyl cellulose or a carboxymethyl cellulose. The conductive gel may include ionic salts from about 50 mEq/L to about 200 mEq/L and in some embodiments about 140 mEq/L. In embodiments, the cannabidiol may be co-solubilized with a protein or a lipophilic surfactant. The lipophilic surfactant may encapsulate the cannabidiol in a liposome.

Additional beneficial effects may arise from including conditioned media recovered from growing cultures of stem cells into the cannabidiol-containing conductive gel. The effects may be two-fold: first cannabidiol has direct effects on the treated tissue at various stages of the healing process. This includes reduction of inflammation, recruitment of endogenous stem cells, and support of differentiation of stem cells into end-stage cells that rebuild or remodel tissue. Second, the cannabidiol may potentiate or enhance the action of the cellular products on the treated tissue.

This specification discloses various aspects of the invention with reference to particular embodiments, but it should be understood that any of the features, functions, materials, or characteristics may be combined with any other of the described features, functions, materials, or characteristics. The description of particular features, functions, materials, or characteristics in connection with a particular embodiment is exemplary only; it should be understood that it is within the knowledge of one skilled in the art to include such feature, structure, or characteristic in connection with other embodiments whether or not explicitly described. We intend the scope of the appended claims to encompass such alternative embodiments. Variations on these described embodiments will become apparent to those of ordinary skill in the art upon reading the description. The inventors expect skilled artisans to employ such variations as appropriate, and the inventors intend for the invention to be practiced otherwise than specifically described herein. Accordingly, this specification and claims include all modifications and equivalents of the subject matter recited in the claims appended hereto as permitted by applicable law.

Unless otherwise indicated, all numbers used in the specification and claims are to be understood as being modified in all instances by the term “about.” Unless indicated to the contrary, the numerical values in the specification and attached claims are approximations that may vary depending upon the desired properties sought to be obtained. The disclosure of each document (including each patent application or patent) described in this document is incorporated by reference herein. In the event of a conflict between this document and the content of documents incorporated by reference, this document shall control.

The terms “a,” “an,” “the” and similar referents used in the context of describing the invention (especially in the context of the following claims) are intended to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the claims. No language in the specification should be construed as indicating any non-claimed element essential to the practice of the invention.

I claim:

1. A dressing for treating a wound, the dressing comprising:
a flexible membrane having an upper surface and a wound-facing surface opposing the upper surface;
a battery affixed to the upper surface;
an electrode pattern to deliver current to the wound, the electrode pattern disposed on the wound-facing surface;
a current control electrically connected to the battery and to the electrode pattern and configured to limit the amount of current delivered to the electrode pattern.

2. The dressing of claim 1, wherein the current control is configured to select the amount of current delivered to the electrode pattern.

3. The dressing of claim 2, wherein the current control includes one of an active circuit, a series resistance control, and a parallel resistance control.

4. The dressing of claim 3, wherein the dressing includes a trim edge, and wherein the current control includes a processor having a plurality of input ports and a plurality of conductors, each electrically connected to a respective one of the plurality of input ports, the plurality of conductors disposed in a spaced-apart relationship parallel to the trim edge.

5. The dressing of claim 3, wherein the current control includes a plurality of resistors arranged in a parallel circuit between the battery and the electrode pattern.

6. The dressing of claim 5, wherein the dressing includes a trim edge and the plurality of resistors are disposed in a spaced-apart relationship parallel to the trim edge.

7. The dressing of claim 3, wherein the current control includes a plurality of resistors arranged in a series circuit between the battery and the electrode pattern.

8. The dressing of claim 7, wherein the upper surface includes a first contact electrically connected to the electrode pattern, a plurality of resistor contacts, each resistor contact electrically connected to a respective one of the plurality of resistors, and a bridging contact configured to electrically connect the first contact to a selected one of the plurality of resistor contacts.

9. The dressing of claim 1, wherein the electrode pattern includes interdigitated electrodes.

10. The dressing of claim 9, wherein the interdigitated electrodes includes a sawtooth interdigitated electrode.

11. A method of treating a wound comprising the steps of:
adjusting the current delivered by the dressing of claim 1; and
applying the dressing to the wound to be treated.

12. The method of claim 11, further comprising readjusting the current delivered by the dressing after the step of applying the dressing to the wound to be treated.

13. The method of claim 12, wherein the dressing includes a trim edge, wherein the current control includes a plurality of resistors arranged in a parallel circuit between the battery and the electrode pattern, wherein the plurality of resistors are disposed in a spaced-apart relationship parallel to the trim edge, and wherein the step of adjusting the current includes cutting the dressing parallel to the trim edge such that one or more of the plurality of resistors are removed from the parallel circuit.

14. The method of claim 12, wherein the current control includes a plurality of resistors arranged in a series circuit between the battery and the electrode pattern, wherein the upper surface includes a first contact electrically connected to the electrode pattern, a plurality of resistor contacts, each resistor contact electrically connected to a respective one of the plurality of resistors, and a bridging contact configured to electrically connect the first contact to a selected one of the plurality of resistor contacts, and wherein the step of adjusting the current includes folding the dressing to electrically connect the bridging contact between the first contact and a selected one of the plurality of resistor contacts.

15. The method of claim 12, wherein the dressing includes a trim edge, wherein the current control includes a processor having a plurality of input ports and a plurality of conductors, each electrically connected to a respective one of the plurality of input ports, the plurality of conductors disposed in a spaced-apart relationship parallel to the trim edge, and wherein the step of adjusting the current includes cutting the dressing parallel to the trim edge such that one or more of the plurality of conductors are severed from the respective one or more of the plurality of input ports.

16. The method of claim 11, further comprising applying a conductive treatment gel including a human adipose-derived stem cell culture (HADSCC) media and a gelling agent between the dressing and the wound.

17. The method of claim 11, further comprising applying a conductive treatment gel including cannabidiol and a gelling agent between the dressing and the wound.

18. A kit comprising:
the dressing of claim 1; and
a conductive gel including an HADSCC media and a gelling agent,
wherein the gel has a viscosity of at least 3000 cP.

19. A kit comprising:
a dressing for treating a wound, the dressing including
a flexible membrane having an upper surface and a wound-facing surface opposing the upper surface; and
an electrode pattern to deliver current to the wound, the electrode pattern disposed on the wound-facing surface; and
a conductive gel including cannabidiol and a gelling agent, wherein the gel has a viscosity of at least 3000 cP.

20. The kit of claim 19, wherein the dressing further includes a current control and a battery, the current control electrically connected to the battery and to the electrode pattern and configured to limit the amount of current delivered to the electrode pattern, and wherein the conductive gel further includes an HADSCC media.
